(19) United States
(12) Patent Application Publication
(10) Pub. No.: US 2010/0222808 A1
(43) Pub. Date: Sep. 2, 2010

(54) TOURNIQUET COUPLING DEVICE

(76) Inventor: James P. Morrow, Renton, WA (US)

Correspondence Address: MCDERMOTT WILL & EMERY LLP, 18191 VON KARMAN AVE., SUITE 500, IRVINE, CA, 92612-7108, US

(21) Appl. No.: 12/695,993

(22) Filed: Jan. 28, 2010

Related U.S. Application Data
(60) Provisional application No. 61/147,977, filed on Jan. 28, 2009.

(51) Int. Cl. A61B 17/132

(52) U.S. Cl. 606/203

(57) ABSTRACT

Certain embodiments provide a tourniquet coupling device that includes a coupling member having first and second end regions, the coupling member including a top wall extending from the first end region to the second end region, a top wall edge; and a bottom wall, coupled to, or integral with, the top wall, that extends from the first to the second end region. In certain embodiments, the bottom wall comprises a bottom wall edge and an opening, and the top and bottom wall edges are separated by a space. In certain embodiments, the coupling member is configured such that, when a first portion of a tourniquet is attached to the coupling member at the first end region, and a second portion of the tourniquet is passed into the opening and into the space, the second end region is attachable to the second portion of the tourniquet.

### RELATED APPLICATIONS

This application claims the benefit of U.S. Provisional Application Ser. No. 61/147,977, filed on Jan. 28, 2009, and titled “Tourniquet Coupling Device”, the entirety of which is hereby incorporated by reference.

### FIELD

Embodiments of the present invention relate to ligature and constriction devices, such as tourniquets, applied to an extremity of an individual to restrict blood flow to and/or from the extremity.

### BACKGROUND

Ligature and constriction devices, such as tourniquets, for restricting blood flow to and/or from an extremity of an individual have wide application in the medical field for the control of blood flow. Typically, a rope, belt, cloth, tubing, or the like is wrapped about a limb of an individual, and then tightened about the limb until blood flow is restricted to and/or from the limb. The restricted blood flow can result in, e.g., enlarging and exposing veins in the limb for the withdrawal of blood, nonbleeding from a wound, and/or a dry surgical field. Tourniquets are used by orthopedists, anesthesiologists, emergency medical technicians, phlebotomists, and other medical practitioners to stop or reduce the flow of blood through an artery by compression.

In certain contexts, such as some surgical practices and some phlebotomy practices, disposable syringes, needles, latex gloves, etc. are used to withdraw blood from subjects, and then discarded in compliance with good medical practice and governmental regulations. The use and regulated disposal of such items are practiced to guard against, e.g., the spread of infectious blood-borne diseases. The risk of transmission of a blood-borne disease in such contexts run from patient to patient, from patient to medical attendant, and from medical attendant to the patient. Single use medical devices, such as disposable syringes, latex gloves, needles, bandages, gauze, cotton swabs, tapes, and the like, that are routinely, and often mandatorily discarded after a single use help minimize the risk of transmission of blood-borne disease. In such contexts, the tourniquet is generally the one re-used item, and its re-use is inconsistent with concerns of blood-borne disease transmission. Good medical practice suggests that the tourniquet should also be a single use device.

### SUMMARY

Prior latex, rubber, or other elastic tourniquet systems are objectionable because of discomforts associated with their application, such as pinching the patient's skin and pulling the patient's body hair. Moreover, the conventional latex tourniquet presents a problem when released as the resulting snapping action can result in the tourniquet contacting the venepuncture site and contaminating the device with blood.

Although the prior art discloses a variety of tourniquets, some disposable, they generally have complicated designs which can make them difficult to use and unsuitable for inexpensive manufacture, and manufacturing costs amount to a significant consideration for disposable devices. Further, many prior art tourniquets are not capable of single-handed application or reapplication.

Certain embodiments of the invention, also known as the Morrow tourniquet coupling device, provide a tourniquet coupling device, for coupling portions of a tourniquet to reduce circulation in an extremity of a patient, comprising: a coupling member having first and second end regions and comprising: a top wall extending from the first end region to the second end region and comprising a top wall edge; and a bottom wall, coupled to, or integral with, the top wall and extending from the first end region to the second end region; wherein the bottom wall comprises a bottom wall edge and an opening extending to the bottom wall edge; and wherein the top wall edge and the bottom wall edge are separated by a space; and wherein the coupling member is configured such that, when a first portion of a tourniquet is attached to the coupling member at the first end region, and a second portion of the tourniquet is passed into the opening and into the space, the second end region is attachable to the second portion of the tourniquet.

In certain embodiments, the top wall comprises a first groove region, comprising a groove and configured to hold at least one of the first and second portions of the tourniquet. In certain embodiments, the first groove region is at the first end region. The top wall may further comprise a first mouth at the first end region. The first mouth may be coextensive with the groove at the first groove region and may be bounded by a first surface and a second surface of the top wall. The first and second surfaces may converge toward one another as they extend from the first end region toward the second end region. In certain embodiments, the first groove region is at the second end region and is configured to hold to the second portion of the tourniquet.

In certain embodiments, the top wall comprises a second groove region located at the first end region, the second groove region comprising a groove configured to hold the second portion of the tourniquet while the first portion of the tourniquet is held by the groove at the second groove region.

In certain embodiments, the groove at the second groove region is coextensive with the groove at the first groove region.

In certain embodiments, the device comprises a side wall located between the top and bottom walls. In certain embodiments, the device further comprises a first beveled edge at a junction of the side wall and the top wall. The first beveled edge, the side wall, and the top wall may be integrally formed. In certain embodiments, the device further comprises a second beveled edge at a junction of the side wall and the bottom wall. The second beveled edge, the side wall, and the bottom wall may be integrally formed.

In certain embodiments, the device comprises a lip at the bottom wall edge, configured to retain the tourniquet between the top and bottom walls.

In certain embodiments, the coupling member is substantially flexible.

In some embodiments, the coupling member further comprises a projection extending from the top wall into the space. In some embodiments, the bottom wall has a surface having an innermost point that is closer to the top wall than is any other point on the surface and the projection extends from the top wall beyond the innermost point. In some embodiments, the projection obstructs a straight path through the space from the first end region to the second end region.

In certain embodiments, the bottom wall comprises one or more cavities. In some embodiments, the bottom wall comprises a substantially planar surface. In some embodiments, the bottom wall comprises an outer surface on the opposite side of the bottom wall from the space and the outer surface is substantially planar.

In some embodiments, the device comprises at least one of a polymer and a thermoplastic. The polymer may comprise an acrylonitrile butadiene styrene polymer.

Certain embodiments of the invention provide a tourniquet system, comprising: a coupling member having first and second end regions and comprising: a top wall extending from the first end region to the second end region and comprising a top wall edge; and a bottom wall, coupled to, or integral with, the top wall and extending from the first end region to the second end region; wherein the bottom wall comprises a bottom wall edge and an opening extending to the bottom wall edge; and wherein the top wall edge and the bottom wall edge are separated by a space; and wherein the coupling member is configured such that, when a first portion of a tourniquet is attached to the coupling member at the first end region, and a second portion of the tourniquet is passed into the opening and into the space, the second end region is attachable to the second portion of the tourniquet; and the tourniquet.

Certain embodiments provide a method, of attaching a tourniquet to an extremity of a patient, comprising: providing a tourniquet, a first portion of which is attached to a first end region of a coupling member; at least partially encircling a patient's extremity with the tourniquet; passing a second portion of the tourniquet into an opening in a bottom wall of the coupling device and into a space between the bottom wall and a top wall of the coupling device, the top wall coupled to, or integral with, the bottom wall; attaching the second portion of the tourniquet to a second end region of the coupling device, so as to reduce circulation in the extremity.

In certain embodiments, the top and bottom walls extend from the first end region to the second end region. In certain embodiments, the attaching comprises inserting the second portion of the tourniquet into a groove in the second end region. In certain embodiments, the first portion of the tourniquet is attached to the coupling member by insertion into a groove in the first end region.

Certain embodiments of the invention provide a tourniquet coupling device, for coupling to a tourniquet and reducing circulation in an extremity of a patient, the device comprising: a tourniquet coupling member configured to couple to a tourniquet and thereby achieve a girdling configuration in which the tourniquet and the member substantially girdle a portion of an extremity of a patient and apply, to the girdled portion of the extremity, constrictive pressure effective to reduce a body fluid circulation in the extremity; wherein the coupling member comprises a top wall having a slot that extends from a first end region of the top wall toward a second end region of the top wall, a bottom wall comprising an aperture, and a first side wall that couples the top and bottom walls; and wherein, when the member and the tourniquet are in the girdling configuration, a first segment of the tourniquet couples to the coupling member; and wherein, when the member and the tourniquet are in the girdling configuration, a section of the tourniquet is positioned at least partially within the aperture and the slot receives a second segment of the tourniquet, thereby securing the coupling member and the tourniquet in the girdling configuration.

In certain embodiments, the slot comprises, at a peripheral region of the top wall, a mouth region characterized by a decrease in width, along an axis of the mouth extending from peripheral to inner regions of the top wall. In certain embodiments, a widest width of the mouth is greater than a width of the second segment in a substantially unfolded form; wherein at least some narrower widths of the mouth are positioned adjacent the slot and are substantially equal to (i) a width of the second segment in a substantially folded form and (ii) a width of the slot, such that, when the second segment is passed through the mouth, in a direction from peripheral to inner regions of the top wall, and into the slot, the second segment assumes the substantially folded form as a result of contact between the second segment and surfaces of the top wall abutting narrower widths of the mouth; and wherein, when the tourniquet and the member are in the girdling configuration, the slot receives the second segment in the substantially folded form such that surface portions of the top wall abutting the slot contact surface portions of the substantially folded second segment, thereby securing the tourniquet and the member in the girdling configuration.

Certain embodiments provide a tourniquet system comprising: a tourniquet coupling member configured to couple to a tourniquet and thereby achieve a girdling configuration in which the tourniquet and the member substantially girdle a portion of an extremity of a patient and apply, to the girdled portion of the extremity, constrictive pressure effective to reduce a body fluid circulation in the extremity; wherein the coupling member comprises a top wall having a slot that extends from a first end region to a second end region of the top wall, a bottom wall comprising an aperture, and a first side wall that couples the top and bottom walls; and wherein, when the member and the tourniquet are in the girdling configuration, a first segment of the tourniquet couples to the coupling member; and wherein, when the member and the tourniquet are in the girdling configuration, a section of the tourniquet is positioned at least partially within the aperture and the slot receives a second segment of the tourniquet, thereby securing the coupling member and the tourniquet in the girdling configuration; and the tourniquet. In certain embodiments, the tourniquet is substantially flexible. In certain embodiments, the tourniquet is substantially flat along the short axis of the tourniquet.

In certain embodiments, when the tourniquet and the member are in the girdling configuration, an edge area of the bottom wall that abuts the aperture makes substantially uniform contact with at least a portion of the section of the tourniquet positioned at least partially within the aperture along the portion's short axis, thereby inhibiting transmission of the substantially folded form of the second segment, along the long axis of the tourniquet, such that contact between the extremity and the tourniquet is substantially uniform along the short axis of the tourniquet.

### BRIEF DESCRIPTION OF THE DRAWINGS

The accompanying drawings, which are included to provide further understanding of the invention and are incorporated in and constitute a part of this specification, illustrate aspects of the invention and together with the description serve to explain the principles of the invention.

FIG. 1 illustrates an example of a tourniquet coupling member, in accordance with various embodiments of the subject disclosure.

FIG. 2 illustrates an example of a tourniquet system, in accordance with various embodiments of the subject disclosure.

FIGS. 3A and 3B illustrate an example of a tourniquet coupling member, in accordance with various embodiments of the subject disclosure.

FIG. 4 illustrates a bottom view of a tourniquet coupling member, in accordance with various embodiments of the subject disclosure.

FIGS. 5A and 5B illustrate a top view of a tourniquet coupling member, in accordance with various embodiments of the subject disclosure.

FIGS. 6A and 6B illustrate side views of a tourniquet coupling member, in accordance with various embodiments of the subject disclosure.

FIG. 7 illustrates a side view of a tourniquet coupling member, in accordance with various embodiments of the subject disclosure.

FIG. 8 illustrates an example of a method of attaching a tourniquet to an extremity of a patient, in accordance with various embodiments of the subject disclosure.

### DETAILED DESCRIPTION

In the following detailed description, numerous specific details are set forth to provide a full understanding of the present invention. It will be apparent, however, to one ordinarily skilled in the art that the present invention may be practiced without some of these specific details. In other instances, well-known structures and techniques have not been shown in detail so as not to obscure the present invention.

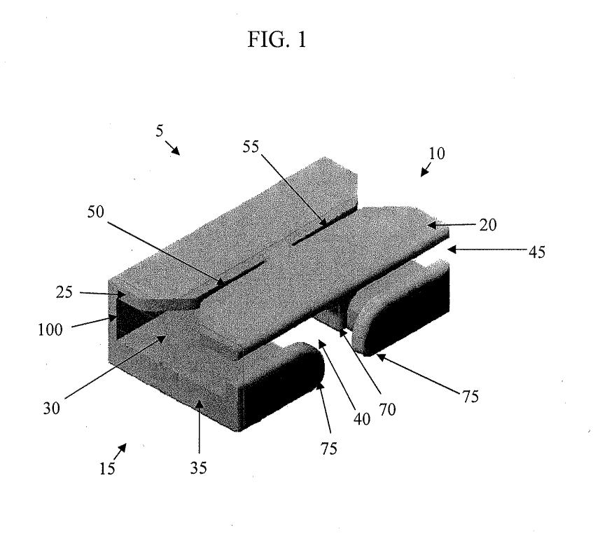

FIG. 1 illustrates an example of a tourniquet coupling member 5, in accordance with various embodiments of the subject disclosure. As shown in FIG. 1, tourniquet coupling member 5 comprises first end region 10 and second end region 15. Top wall 20 extends from first end region 10 to second end region 15 and comprises top wall edge 25. Bottom wall 30 is coupled to top wall 20 and extends from first end region 10 to second end region 15, and bottom wall 30 comprises bottom wall edge 35 and opening 40 running to the bottom wall edge. As shown in FIG. 1, top wall edge 25 and bottom wall edge 35 are separated by space 45; and coupling member 5 is configured such that, when a first portion of a tourniquet (not shown) is attached to coupling member 5 at the first end region 10, and a second portion of the tourniquet (not shown) is passed into opening 40 and into the space 45, the second end region is attachable to the second portion of the tourniquet (not shown). In some embodiments, the coupling member 5 comprises a side wall 100 located between the top wall 20 and the bottom wall 30.

As shown in FIG. 1, top wall 20 comprises first groove region 50, which is configured to hold at least one of the first and second portions of the tourniquet (not shown). As shown, first groove region 50 is at second end region 15 and is configured to attach to the second portion of the tourniquet. In addition, top wall 20 comprises second groove region 55 at first end region 10, such that when the first portion of the tourniquet (not shown) is held by the second groove region 55, first groove region 50 is configured to hold the second portion of the tourniquet (not shown).

In certain embodiments, the coupling member 5 further comprises a projection 70 extending from the top wall 20 into the space 45. In certain embodiments, the coupling member 5 further comprises a lip 75 at the bottom wall edge 35, configured to retain the tourniquet between the top wall 20 and the bottom wall 30.

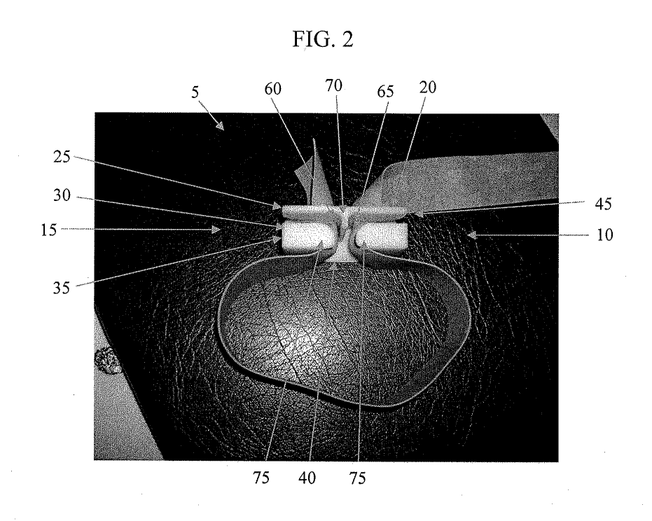

FIG. 2 illustrates an example of a tourniquet coupling member 5, in accordance with various embodiments of the subject disclosure. As shown in FIG. 2, tourniquet coupling member 5 comprises first end region 10 and second end region 15. Top wall 20 extends from first end region 10 to second end region 15 and comprises top wall edge 25. Bottom wall 30 is coupled to top wall 20 and extends from first end region 10 to second end region 15, and bottom wall 30 comprises bottom wall edge 35 and opening 40 running to the bottom wall edge. As shown in FIG. 2, top wall edge 25 and bottom wall edge 35 are separated by space 45; and coupling member 5 is configured such that, when first portion of tourniquet 60 is attached to coupling member 5 at the second end region 15, and a second portion of the tourniquet 65 is passed into opening 40 and into the space 45, first end region 10 is attachable to the second portion of the tourniquet 65. In some embodiments, the projection 70 obstructs a straight path through the space 45 from the first end region 10 to the second end region 15.

In some embodiments, a first and a second groove can be continuous from first end region 10 to second end region 15 of the member 5. In some embodiments, first and second grooves can be discontinuous. In some embodiments, a tourniquet adapter member can secure a tourniquet in a girdling configuration around an extremity of a patient by a friction fit. In certain embodiments, the coupling member 5 can comprise at least one of a polymer, a thermoplastic, and other suitable materials known to those of ordinary skill in the art. In some embodiments, the polymer comprises an acrylonitrile butadiene styrene (ABS) polymer. For example, the polymer may comprise ABS Polylac PA-757.

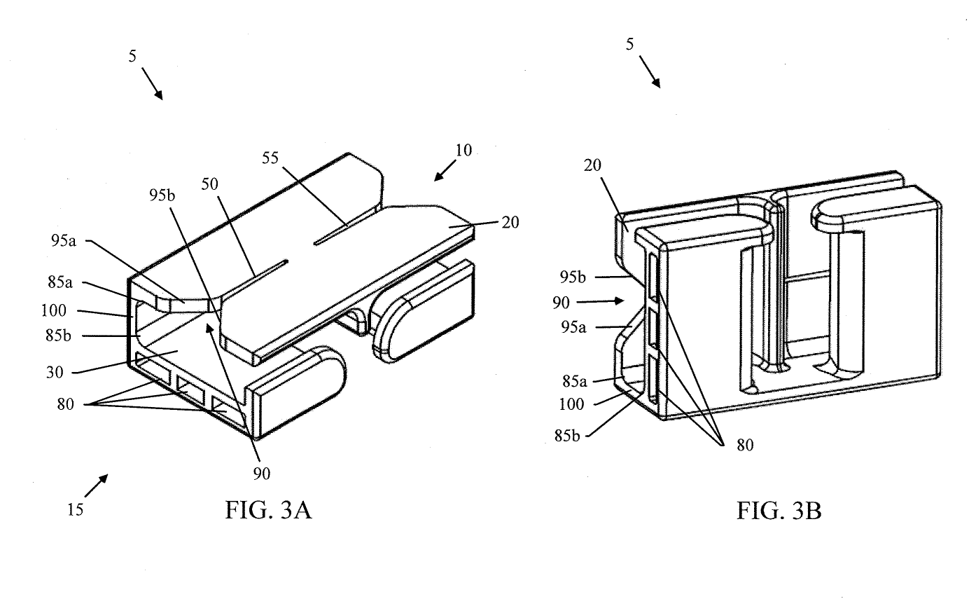

FIGS. 3A and 3B illustrate an example of a tourniquet coupling member 5, in accordance with various embodiments of the subject disclosure. In certain embodiments, the top wall 20 further comprises a mouth 90 that is coextensive with a groove of a groove region (e.g., groove region 50 or groove region 55) and is bounded by a first surface 95a and a second surface 95b of the top wall 20. The first surface 95a and the second surface 95b converge toward one another as they extend from one end region (e.g., first end region 10 or second end region 15) toward the other end region of the coupling member 5.

In some embodiments, the coupling member 5 comprises a first beveled edge 85a at a junction of the side wall 100 and the top wall 20. In certain embodiments, the first beveled edge 85a, the side wall 100, and the top wall 20 are integrally formed. The first beveled edge 85a may provide additional structural support between the side wall 100 and the top wall 20. According to certain embodiments, the coupling member 5 comprises a second beveled edge 85b at a junction of the side wall 100 and the bottom wall 30. In some embodiments, the second beveled edge 85b, the side wall 100, and the bottom wall 30 are integrally formed. The second beveled edge 85b may provide additional structural support between the side wall 100 and the bottom wall 30.

In certain embodiments, the bottom wall 30 comprises one or more cavities 80. The one or more cavities 80 may allow the coupling member 5 to be lighter compared to a coupling member 5 without the one or more cavities 80. In some embodiments, the one or more cavities 80 allow for bending or structural strengthening of the bottom wall 30 of the coupling member 5.

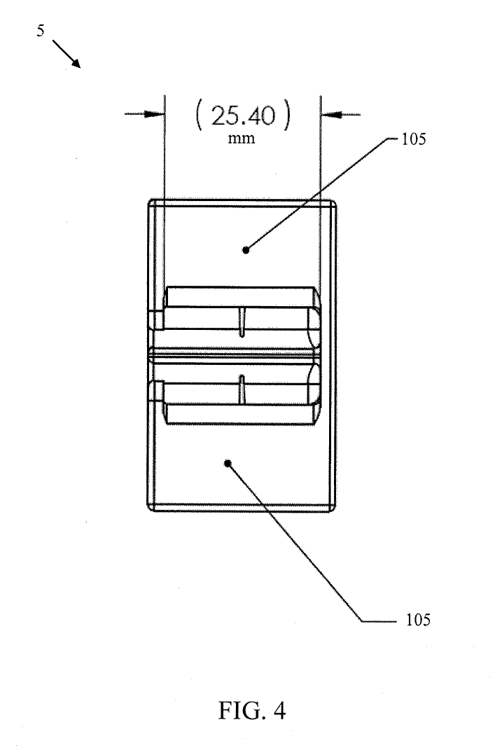

FIGS. 4, 5A, 5B, 6A, 6B, and 7 illustrate various view of a tourniquet coupling member 5, in accordance with various embodiments of the subject disclosure. In some embodiments, these figures provide exemplary dimensions in millimeters (mm) of the coupling member 5. In some embodiments, a radius of curvature is provided for certain dimensions and is indicated by “radial” following the exemplary dimension in mm. In some embodiments, exemplary angles of certain dimensions are provided in degrees. The coupling member 5 is not limited to the dimensions as shown in these figures, and it would be apparent to one of ordinary skill in the art that the coupling member 5 may be fabricated with other suitable dimensions.

FIG. 4 illustrates a bottom view of a tourniquet coupling member 5, in accordance with various embodiments of the subject disclosure. In some embodiments, the bottom wall 30 comprises a substantially planar surface 105. In some embodiments, the bottom wall 30 comprises an outer surface 105 on the opposite side of the bottom wall 30 from the space 45. The outer surface 105 may be substantially planar, in accordance with various embodiments of the subject disclosure.

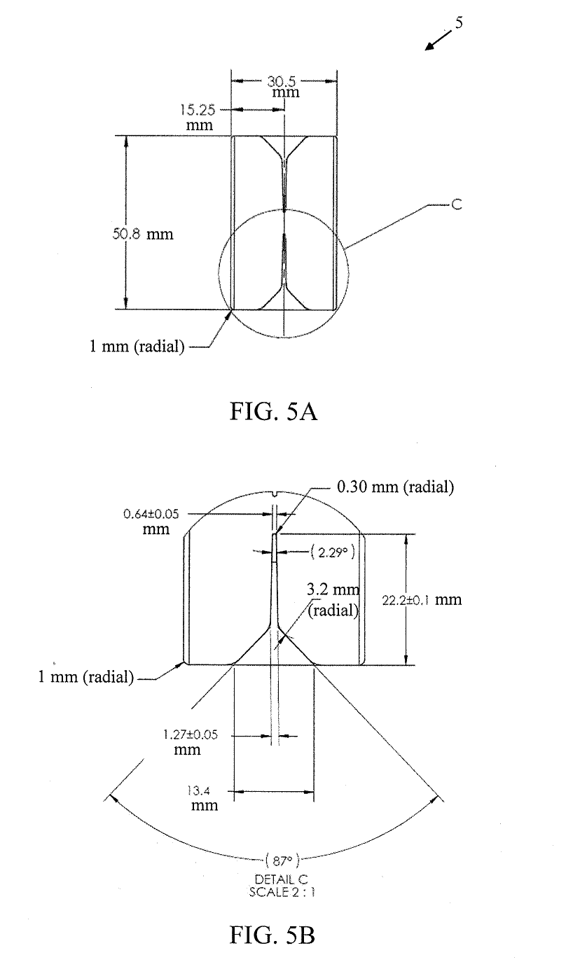

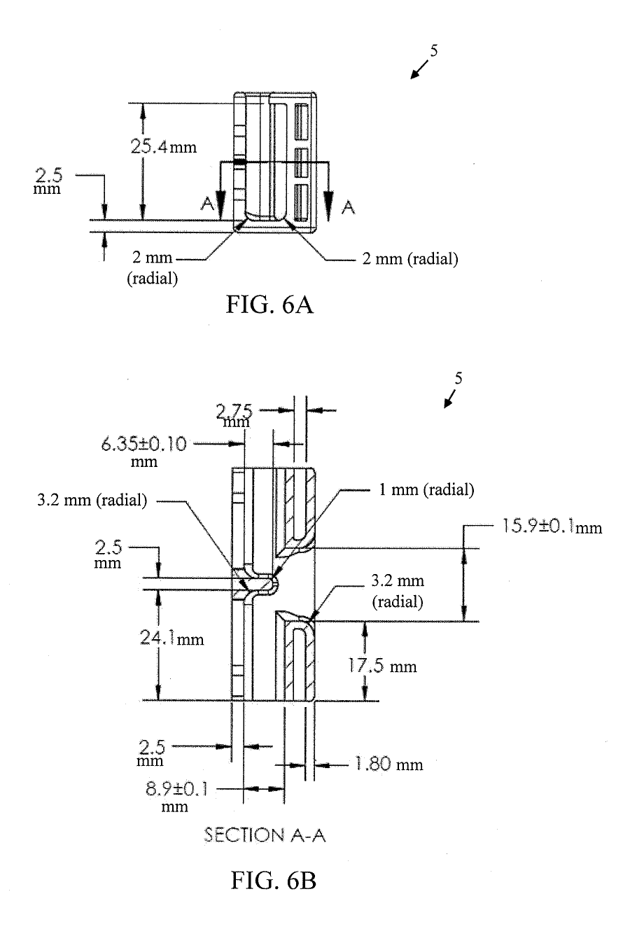

FIGS. 5A and 5B illustrate a top view of a tourniquet coupling member 5, in accordance with various embodiments of the subject disclosure. FIG. 5B provides a detailed view of section C of FIG. 5A. FIGS. 6A and 6B illustrate side views of a tourniquet coupling member 5, in accordance with various embodiments of the subject disclosure. FIG. 6B provides an alternative side view of the coupling member 5, as viewed from section A-A of FIG. 6A.

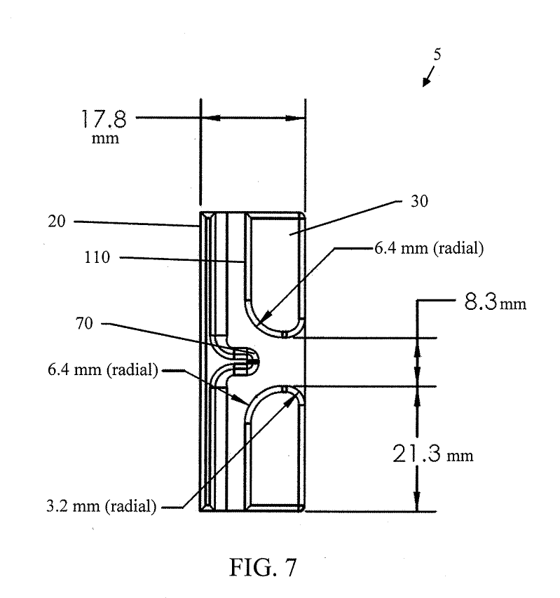

FIG. 7 illustrates a side view of a tourniquet coupling member 5, in accordance with various embodiments of the subject disclosure. In certain embodiments, the bottom wall 30 has a surface 110 having an innermost point that is closer to the top wall 20 than is any other point on the surface 110. According to some aspects, the projection 70 may extend from the top wall 20 beyond the innermost point of the surface 110, as shown in FIG. 7.

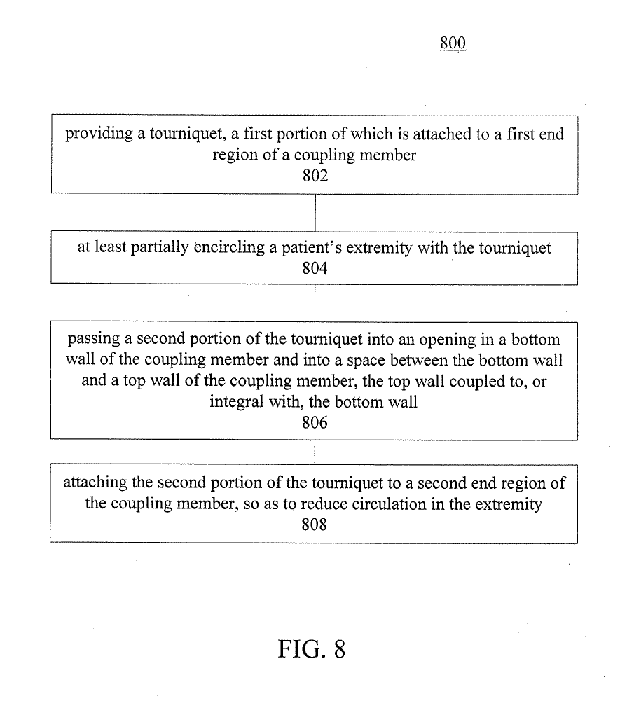

FIG. 8 illustrates an example of a method 800 of attaching a tourniquet to an extremity of a patient, in accordance with various embodiments of the subject disclosure. Method 800 comprises providing a tourniquet, a first portion of which is attached to a first end region of a coupling member (802) and at least partially encircling a patient's extremity with the tourniquet (804). Method 800 also comprises passing a second portion of the tourniquet into an opening in a bottom wall of the coupling member and into a space between the bottom wall and a top wall of the coupling member, the top wall coupled to, or integral with, the bottom wall (806). Method 800 also comprises attaching the second portion of the tourniquet to a second end region of the coupling member, so as to reduce circulation in the extremity (808).

As used herein, the term “groove” has its ordinary meaning, and in addition, can mean, e.g., a cut or indentation in a surface or a slot or slit in an object.

The skilled artisan will recognize the interchangeability of various features and/or steps from different embodiments. Similarly, various of the above-discussed features or steps, as well as other known equivalents for each such feature or step, can be mixed and matched by one of ordinary skill in this art to perform compositions or methods in accordance with principles described herein. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

What is claimed is:

1. A tourniquet coupling device, for coupling portions of a tourniquet to reduce circulation in an extremity of a patient, comprising:
a coupling member having first and second end regions and comprising:
a top wall extending from the first end region to the second end region and comprising a top wall edge; and
a bottom wall, coupled to, or integral with, the top wall and extending from the first end region to the second end region;
wherein the bottom wall comprises a bottom wall edge and an opening extending to the bottom wall edge;
wherein the top wall edge and the bottom wall edge are separated by a space;
wherein the coupling member is configured such that, when a first portion of a tourniquet is attached to the coupling member at the first end region, and a second portion of the tourniquet is passed into the opening and into the space, the second end region is attachable to the second portion of the tourniquet to reduce circulation in the extremity.

2. The device of claim 1, wherein the top wall comprises a first groove region, comprising a groove configured to hold at least one of the first and second portions of the tourniquet.

3. The device of claim 2, wherein the first groove region is at the first end region, wherein the top wall further comprises a first mouth at the first end region, wherein the first mouth is coextensive with the groove at the first groove region and is bounded by a first surface and a second surface of the top wall, and wherein the first and second surfaces converge toward one another as they extend from the first end region toward the second end region.

4. The device of claim 2, wherein the first groove region is at the second end region and is configured to hold to the second portion of the tourniquet.

5. The device of claim 4, wherein the top wall comprises a second groove region located at the first end region, the second groove region comprising a groove configured to hold the second portion of the tourniquet while the first portion of the tourniquet is held by the groove at the second groove region.

6. The device of claim 5, wherein the groove at the second groove region is coextensive with the groove at the first groove region.

7. The device of claim 1, further comprising a side wall located between the top and bottom walls.

8. The device of claim 7, further comprising a first beveled edge at a junction of the side wall and the top wall.

9. The device of claim 1, further comprising a lip at the bottom wall edge, configured to retain the tourniquet between the top and bottom walls.

10. The device of claim 1, wherein the coupling member is substantially flexible.

11. The device of claim 1, wherein the coupling member further comprises a projection extending from the top wall into the space.

12. The device of claim 11, wherein the bottom wall has a surface having an innermost point that is closer to the top wall than is any other point on the surface, and wherein the projection extends from the top wall beyond the innermost point.

13. The device of claim 1, wherein the bottom wall comprises one or more cavities.

14. The device of claim 1, further comprising at least one of a polymer and a thermoplastic.

15. The device of claim 14, wherein the polymer comprises an acrylonitrile butadiene styrene polymer.

16. A tourniquet system, comprising:
the device of claim 1; and
the tourniquet.

17. A method, of attaching a tourniquet to an extremity of a patient, comprising:
providing a tourniquet, a first portion of which is attached to a first end region of a coupling member;
at least partially encircling a patient's extremity with the tourniquet;
passing a second portion of the tourniquet into an opening in a bottom wall of the coupling member and into a space between the bottom wall and a top wall of the coupling member, the top wall coupled to, or integral with, the bottom wall;
attaching the second portion of the tourniquet to a second end region of the coupling member, so as to reduce circulation in the extremity.

18. The method of claim 17, wherein the top and bottom walls extend from the first end region to the second end region.

19. The method of claim 17, wherein the attaching comprises inserting the second portion of the tourniquet into a groove in the second end region.

20. The method of claim 19, wherein the first portion of the tourniquet is attached to the coupling member by insertion into a groove in the first end region.
